(19) United States
(12) Patent Application Publication
(10) Pub. No.: US 2003/0072815 A1
(43) Pub. Date: Apr. 17, 2003

(54) Reverse

(76) Inventors: Belinda Jean Rudder, Roosevelt, NY (US); Erton Wendell Rudder JR., Roosevelt, NY (US); Ebony Shanee Rudder, Roosevelt, NY (US)

Correspondence Address: Belinda Rudden, 256 Lincoln Ave., Roosevelt, NY, 11575, US

(21) Appl. No.: 09/975,130

(22) Filed: Oct. 12, 2001

(51) Int. Cl. A61K 35/78

(52) U.S. Cl. 424/725

(57) ABSTRACT

A injection dosage form containing pure peanut oil in controlling disorders that arise in cells, or correct errors in the genetic code that cause mental, and physical disorders also in the process Mitosis cells divide, or forms identical cells, restore cells hereditary material that's scattered throughout the nucleus, restore aging, DNA restore torn tissue, muscle, alter the code and erase hundreds of inherited mental, and physical aids, deadly diseases, by enlarge cells, asthma, heart problems, child bearing, enlarge nose, lip, breast, penis, ear lobe, hearing, lungs, veins, gallstones, alter mature nerve cells, reproduce, or replace damage, or destroyed cells, replace worn out, or disease tissue, muscles, heart, cataracts, a stump of an amputated limb to re-grow. This will change the hereditary process of humans with diseases. These disorders are provided in an acceptable form. The dosage form comprises pure peanut oil which is applies in an amount that provides for a relatively period during which the composition is being excepted by a patient, but which allows substantially immediate release of the pure peanut oil after the composition has been injected. This will correct diseases that result from foreign invader organisms call pathogens, also that threaten our homeostasis-identified molecules or outer surfaces the membrane.

The development of DNA technology and a good function gene can be inserted into the cells of a patient, and can correct genetic defects, also give new function to the cell. This is the way type of viruses attack humans cells. The adjacent tissue this cell is the basic building block fundamental unit of the body chromosomes of cell membranes and the nucleus, cytoplasm within the cell membrane and the nucleus holds the genetic material chromosomes this will cure all defects, disorders, diseases of all kind, like cataracts and cancer.

It is an object that is from the plant that grows peanuts. That improves method of enlarging cells. Pure peanut oil.

It is a further object of the invention to provide an improved method for rapidly delivering injections to the adjacent tissues basic building block fundamental unit, chromosomes of cell membrane, cytoplasm.

It is further object of the invention within the cell membrane, nucleus holds the genetic material chromosomes, and homeostasis identified molecules or outer surfaces the membrane.

Briefly, the invention relates to pure peanut oil being injected into the cells of a patient, correct genetic defects, new function, genetically suppress diseases, defected cells, defected genes to protect normal cells, genes, take a of pure peanut oil to adjacent tissues, cells, chromosomes of cell membrane, nucleus, cytoplasm.

There are 23 paired chromosomes in each cell nucleus of humans one pair determines the sex of hereditary material the chromatin scattered throughout the nucleus. Throughout the nucleus this will restore replace worn-out or diseased tissue, joints, muscles, cells, stump of an amputated limb to re-grow, will change hereditary process, slow aging, DNA genetic code cells that cause mental, physical disorders, will increases man's life span. This will alter the code and erase so many inherited mental and physical defects. Reverse cancer, aids, heart disorders, and diseases of all kinds lungs diseases disorder, enlarge nose, lip, breast, penis, ear lope, veins, buttock, alter mature nerve cells, or reproduce, and replace damaged or destroyed cells, child learning bearing difficulties, eyes.

What is claimed I out of 28:

1. We claim as our invention an injection to damaged cells, or immune systems will enlarge increasing the size of the cell of the immune system. Injection dosage form containing pure peanut oil will cure:
I. Disorders nourish the immune system.
II. Body receive imparts good mechanical strength, vitamins in this respect pure peanut oil is superior to other methods that have fell short on finding cures.
III. Genetic codes.
IV. DNA
V. Restore torn tissue.
VI. Erase hundreds of inherited mental and physical defects
VII. Reverse cancer
VIII. Reverse AIDS
IX. Reverse deadly diseases.
X. Enlarging cells
XI. Longer life
XII. Restore asthma
XIII. Heart problems and diseases
XIV. Women with child bearing problems
XV. Enlarge nose
XVI. Enlarge Lip
XVII. Enlarge breast
XVIII. Enlarge penis
XIX. Enlarge ear lobe, or ear size, and hearing
XX. Lungs
XXI. Veins
XXII. Gallstones patients receive relief
XXIII. Alter mature nerve cells, reproduce, and replace damaged or destroyed cells
XXIV. Replace worn-out, or diseased tissue, muscles, also the stump of an amputated limb to re-grow
XXV. Change hereditary process to free farm crops, and animals of diseases
XXVI. Genetically suppress cancer, or defected cells, genes to protect normal cells, genes
XXVII. Adjacent tissues is the cell basic building block fundamental unit inject pure peanut oil
XXVIII. Correct eye sight
